(19) United States
(12) Patent Application Publication
(10) Pub. No.: US 2021/0259678 A1
(43) Pub. Date: Aug. 26, 2021

(54) LAPAROSCOPIC DEVICE WITH SUTURE CUTTER

(71) Applicant: Activ Surgical, Inc., Boston, MA (US)

(72) Inventors: Liam O'Shea, Westwood, MA (US); Thomas J. Calef, Boston, MA (US); Dana Zitnick, Warwick, RI (US)

(21) Appl. No.: 17/191,407

(22) Filed: Mar. 3, 2021

Related U.S. Application Data
(63) Continuation of application No. PCT/US2019/050311, filed on Sep. 10, 2019.
(60) Provisional application No. 62/729,082, filed on Sep. 10, 2018.

(51) Int. Cl. A61B 17/04; A61B 34/30

(52) U.S. Cl. CPC A61B 17/0469; A61B 17/0467; A61B 2034/301; A61B 34/30; A61B 2017/0474; A61B 17/0493

(57) ABSTRACT

A laparoscopic device with suture cutter includes a tubular housing having first, second, and third channels. Each channel extends longitudinally along the housing. A first suture grasping instrument in the first channel has an end effector that can be actuated to extend outwardly from the distal end of the housing to grasp a first portion of a suture. A second suture grasping instrument in the second channel has an end effector that can be actuated to extend outwardly from the distal end to grasp a second portion of a suture. A cutting blade is recessed in the distal end at a location between the first and second channels. The first and second instruments can be retracted into the distal end of the housing while grasping the suture and to engage the suture with the blade to sever the suture. The third channel can house a laparoscope.

### CROSS REFERENCE TO RELATED APPLICATION

This application is a continuation application of International Application No. PCT/US2019/050311, filed on Sep. 10, 2019, which application claims priority from U.S. Provisional Patent Application No. 62/729,082, filed on Sep. 10, 2018, which applications are incorporated herein by reference in their entirety for all purposes.

### BACKGROUND

The present application relates generally to surgical devices for suturing body tissue and, more particularly, to devices for cutting sutures during surgery.

Suturing is used to close the edges of a wound or incision and to repair damaged tissue during surgery. Sutures can be applied using a suturing device having a needle, which can be manipulated to draw a suture through the tissue at the edges of the incision. The suture can be tightened to draw the edges of the tissue together, and then knotted and the suture tail trimmed.

### BRIEF SUMMARY

A laparoscopic device with a suture cutter is disclosed in accordance with one or more embodiments. The device includes an elongated tubular housing having a proximal end and an opposite distal end. The housing has first and second working channels therein. Each channel extends longitudinally along the length of the tubular housing. A first suture grasping instrument in the first channel of the housing has an end effector that can be actuated to extend outwardly from the distal end of the housing to grasp a first portion of a suture. A second suture grasping instrument in the second channel of the housing has an end effector that can be actuated to extend outwardly from the distal end of the housing to grasp a second portion of a suture. A cutting blade is recessed in the distal end of the housing at a location between the first and second channels. The first and second suture grasping instruments can be actuated to be retracted into the distal end of the housing while grasping the first and second portions of the suture and to engage the suture with the cutting blade to sever the suture between the first and second portions of the suture.

A method in accordance with one or more embodiments of cutting a suture using a robotically controlled suture cutting device includes the steps of: (a) extending a first suture grasping instrument and a second suture grasping instrument axially outward from a tubular housing of the suture cutting device; (b) grasping a first portion of a suture using the first suture grasping instrument; (c) grasping a second portion of the suture using the second suture grasping instrument; and (d) retracting the first and second suture grasping instruments into the tubular housing such that the suture engages a blade recessed in the tubular housing between the first and second suture grasping instruments to sever the suture.

A robotic suturing system in accordance with one or more embodiments includes a first robotic arm, a second robotic arm, a suturing device operatively coupled to the first robotic arm, a suture manipulator/cutter device operatively coupled to the second robotic arm, and a control system. The control system is operatively coupled to the first robotic arm, the second robotic arm, the suturing device, and the suture manipulator/cutter device to control operation thereof to perform a suturing procedure on a patient. The suture manipulator/cutter device includes an elongated tubular housing having a proximal end and an opposite distal end. The housing has first and second channels therein. Each channel extends longitudinally along the length of the tubular housing. A first suture grasping instrument in the first channel of the housing has an end effector that can be actuated to extend outwardly from the distal end of the housing to grasp a first portion of a suture. A second suture grasping instrument in the second channel of the housing has an end effector that can be actuated to extend outwardly from the distal end of the housing to grasp a second portion of a suture. A cutting blade is recessed in the distal end of the housing at a location between the first and second channels. The first and second suture grasping instruments can be actuated to be retracted into the distal end of the housing while grasping the first and second portions of the suture and to engage the suture with the cutting blade to sever the suture between the first and second portions of the suture.

### BRIEF DESCRIPTION OF THE DRAWINGS

FIG. 1 is a simplified block diagram illustrating an exemplary suturing system in accordance with one or more embodiments.

FIGS. 2 and 3 illustrate operation of the suturing system in a suturing procedure in accordance with one or more embodiments.

FIG. 4 illustrate an exemplary jaw assembly of a grasping instrument in the suturing system in accordance with one or more embodiments.

FIG. 5 illustrates the process of cutting a suture using a suture cutter in the suturing system in accordance with one or more embodiments.

FIGS. 6A and 6B illustrate a suture manipulator/cutter device in accordance with one or more embodiments with suture grasping instruments in extended and retracted positions, respectively.

FIG. 7 is a cross-section view of the suture manipulator/cutter device in accordance with one or more embodiments.

FIG. 8 is another cross-section view the suture manipulator/cutter device in accordance with one or more embodiments.

### DETAILED DESCRIPTION

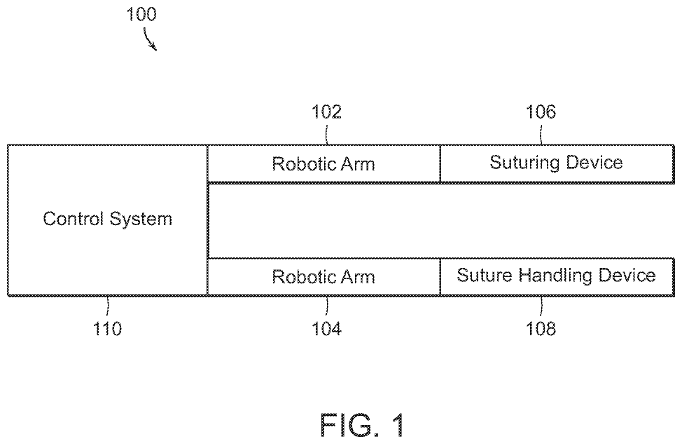

FIG. 1 is a block diagram schematically illustrating one example of a robotic suturing system 100 in accordance with one or more embodiments. The robotic suturing system is used to perform a suturing procedure on a patient lying on an operating table during surgery.

The system includes a first robotic arm 102 operatively coupled to a suturing device 106. The system also includes a second robotic arm 104 operatively coupled to a suture manipulator/cutter device 108. Each robot arm is connected to a computer control system 110, which controls movement of the robot arms and operation of the suturing device 106 and the suture manipulator/cutter device 108.

Examples of robotic arms that can be configured for use in the suturing system include those available from KUKA Robotics, ABB Robotics, DLR Robotics, among others.

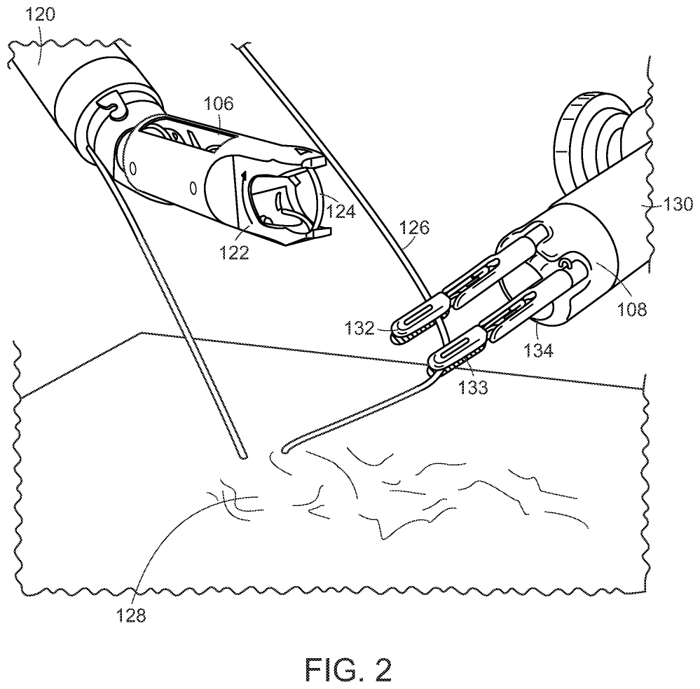

FIG. 2 illustrates operation of the suturing device 106 and the suture manipulator/cutter device 108 in a suturing procedure. The suturing device 106 includes a tubular housing 120 having a proximal end (not shown) coupled to the first robotic arm 102 and an opposite distal end having a suturing end effector 122. The suturing end effector 122 includes a curved needle 124 that can be manipulated to draw a suture 126 through tissue 128. The suture manipulator/cutter device 108 works cooperatively with the suturing device 106 to place and knot the suture 126 in the tissue 128 as shown in FIGS. 2 and 3.

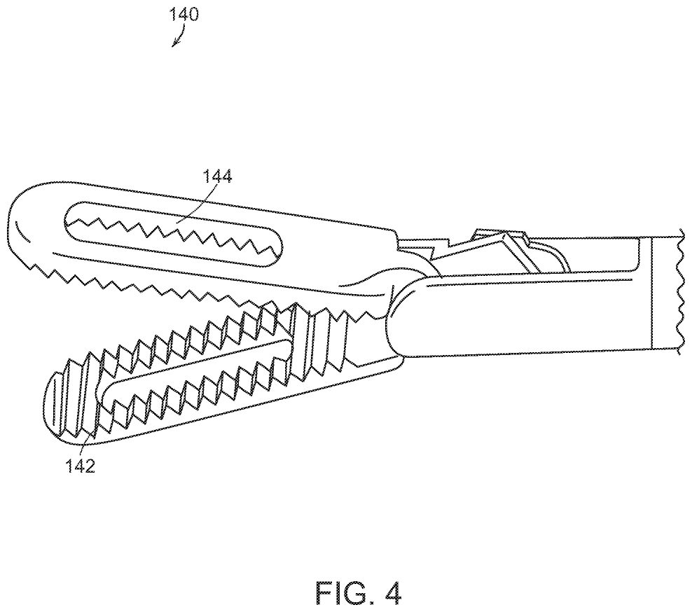

The suture manipulator/cutter device 108 includes an elongated tubular housing 130 having a proximal end (not shown) connected to the second robotic arm 104 and an opposite distal end (shown in FIGS. 2 and 5) having first and second suture grasping end effectors 132, 133 and a laparoscope 134.

The elongated tubular housing 130 includes three internal working channels extending longitudinally along the length of the tubular housing 130. The first and second channels house the first and second suture grasping instruments 132, 133, respectively. The third channel houses the laparoscope 134.

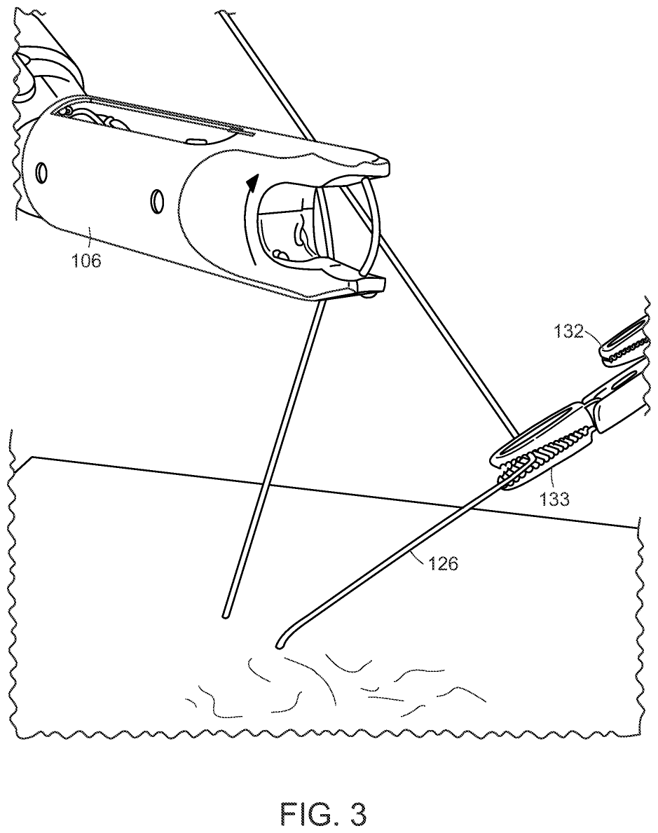

Each grasping instrument 132, 133 includes a jaw assembly 140 (shown in FIG. 4) comprising a pair of opposing jaws 142, 144, which are relatively movable between an open position (as illustrated in FIG. 4) and a closed position (as illustrated in FIG. 2), in which a suture can be grasped.

By way of non-limiting example, each jaw assembly 140 can be a 2 mm to 3.5 mm surgical grasper. The outer diameter of the tubular housings 120, 130 can be, e.g., 15 mm.

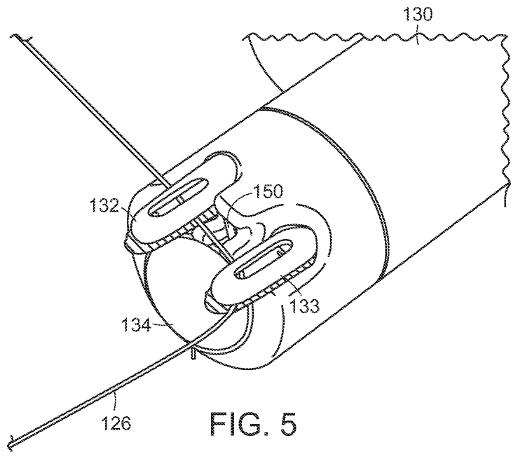

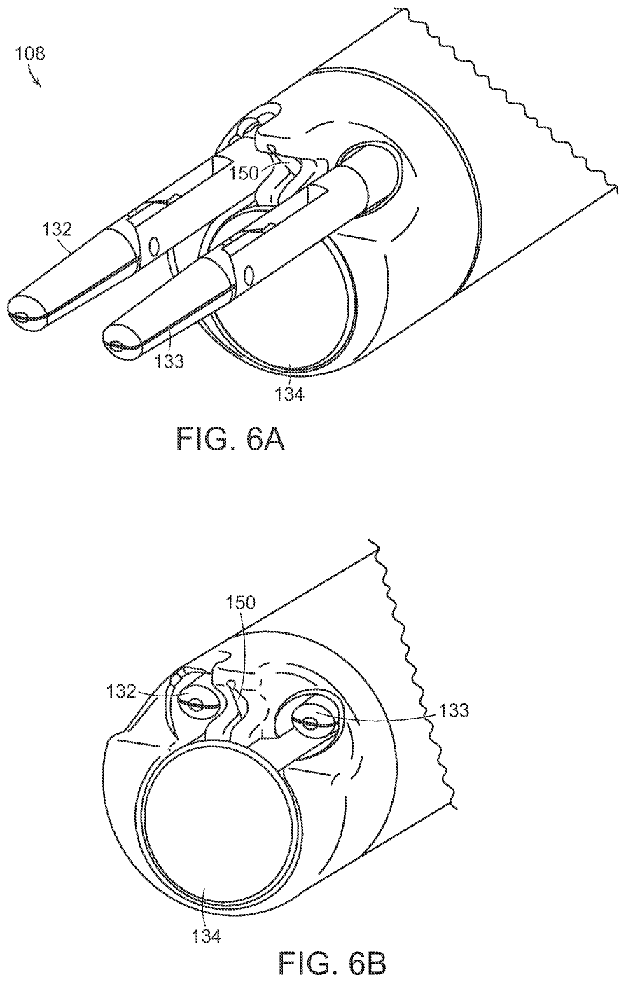

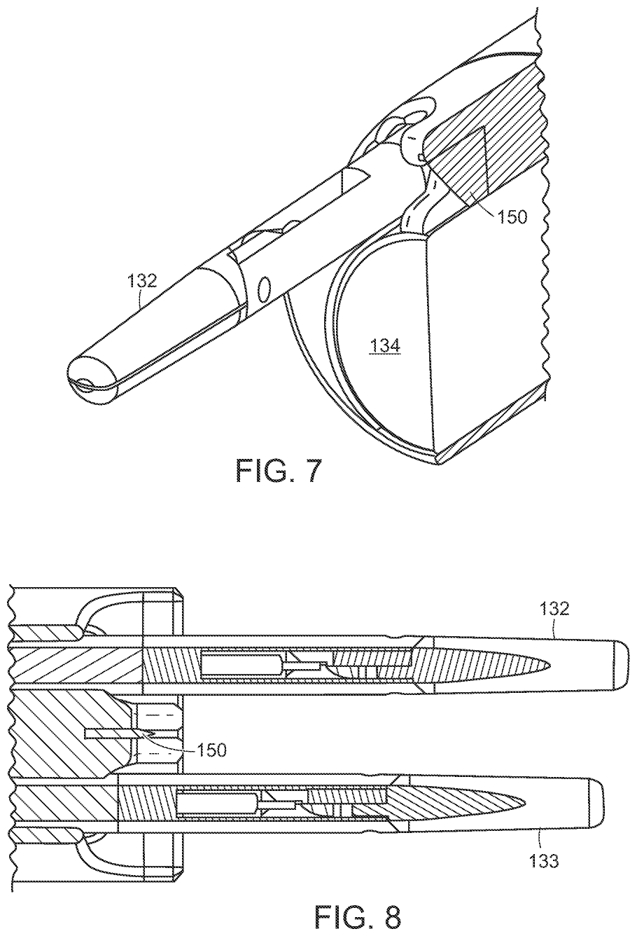

FIGS. 6A and 6B further illustrate the suture manipulator/cutter device 108 with the suture grasping instruments 132, 133 in extended and retracted positions, respectively. FIGS. 7 and 8 are cross-section views of the suture manipulator/cutter device 108. As shown, a suture cutting blade 150 is recessed in the distal end of the housing 130 at a location between the first and second suture grasping instruments 132, 133. As a safety measure in accordance with one or more embodiments, the blade 150 is recessed within the distal end of the housing 130, thereby guarding against damage from inadvertent contact between the blade 150 and the patient or other equipment.

Movement of the first and second suture grasping instruments 132, 133 is controlled by actuators at the proximal end of the devices 132, 133. The jaw assembly 140 of each suture grasping instruments 132, 133 can be controlled to extend axially from or into the distal end of the housing 130 between a retracted position (e.g., shown in FIGS. 5 and 6B) and an extended position (e.g., shown in FIGS. 2 and 6A).

FIGS. 2 and 5 illustrate a process for cutting suture in accordance with one or more embodiments. First, the jaw assemblies 140 of both grasping instrument 132, 133 are extended axially out of the housing 130 and moved by the robotic arm to a position where they each grasp a portion of the suture 126 (FIG. 2). The jaw assemblies 140 grasp the suture 126 at spaced-apart locations on the suture. Next, the jaw assemblies 140 of the suture grasping instruments 132, 133 are actuated to be retracted into the distal end of the housing 130 while grasping portions of the suture (FIG. 5). This retraction of the devices forces the portion of the suture 126 between the grasping instrument 132, 133 to engage and ride along the blade 150, causing the suture to be cut.

The suturing device can be utilized in a variety of robotic surgical equipment, including autonomous robotic surgical systems as well as surgeon-controlled robotic equipment.

One advantage of the suture cutter device integrated in the device 108 having suture graspers is that it avoids the need for a separate device to cut sutures. In other words, the same device 108 used to manipulate suture and work cooperatively with the suturing device 106 to place and knot the suture 126 in the tissue 128, is also used to trim the suture when needed.

In addition, the suture cutter device enables sutures to be quickly and easily grasped and cut.

In accordance with one or more embodiments, suture cutter device enables the tail-side of a suture to be cut, leaving enough suture remaining on the needle-side to allow additional suturing after a knot is tied.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1.-17. (canceled)

18. A suture cutting device, comprising:
a housing comprising a plurality of channels;
a plurality of suture graspers positioned within the plurality of channels, wherein the plurality of suture graspers are extendable from the housing to grasp different portions of a suture; and
a cutting blade recessed in the housing and positioned between the plurality of channels, wherein the plurality of suture graspers are configured to retract into the housing while grasping the different portions of the suture to place the suture in contact with the cutting blade to sever the suture.

19. The device of claim 18, wherein the cutting blade is configured to sever the suture in a region between the different portions of the suture.

20. The device of claim 18, wherein the plurality of suture graspers comprise a jaw assembly comprising a pair of opposing jaws that are relatively movable between an open position and a closed position to grasp the suture.

21. The device of claim 18, wherein the cutting blade comprises a cutting edge that is angled relative to the housing.

22. The device of claim 18, wherein the cutting blade is configured to cut a tail-side of the suture to leave a portion of the suture remaining on a needle-side of the suture to allow for additional suturing after a knot is tied using the suture.

23. A suturing system, comprising:
the suture cutting device of claim 18; and
a scope operably coupled thereto.

24. The system of claim 23, wherein the scope is positioned within a channel of the plurality of channels.

25. A suturing system, comprising:
the suture cutting device of claim 18; and
one or more robotic arms, wherein the suture cutting device is coupled to the one or more robotic arms.

26. The system of claim 25, further comprising a controller operatively coupled to the one or more robotic arms to control an operation or a movement of the one or more robotic arms.

27. The system of claim 26, wherein the controller is configured to control an operation or a movement of the plurality of suture graspers.

28. The system of claim 26, wherein the controller is configured to independently control or operate each of the plurality of suture graspers.

29. The system of claim 26, wherein the controller is configured to autonomously control the operation or the movement of the one or more robotic arms.

30. The system of claim 26, further comprising one or more actuators configured to control an operation or a movement of the plurality of suture graspers.

31. The system of claim 30, wherein the one or more actuators are configured to control the plurality of suture graspers to axially extend from or retract into the housing.

32. The system of claim 25, wherein the one or more robotic arms comprise a first robotic arm comprising a suturing device and a second robotic arm comprising a suture manipulation device.

33. The system of claim 32, wherein the suturing device comprises a curved needle that is configured to draw a suture through a tissue.

34. The system of claim 32, wherein suture manipulation device comprises the suture cutting device.

35. The system of claim 32, further comprising a controller operatively coupled to the first robotic arm, the second robotic arm, the suturing device, and the suture manipulation device.

36. The system of claim 35, wherein the controller is configured to control an operation of the first robotic arm, the second robotic arm, the suturing device, and the suture manipulation device to cooperatively perform a suturing procedure on a patient.

37. The system of claim 36, wherein the suturing procedure comprises placing or knotting a suture in a tissue.
